(19) United States
(12) Patent Application Publication
(10) Pub. No.: US 2014/0255315 A1
(43) Pub. Date: Sep. 11, 2014

(54) ACF DETECTION METHOD

(71) Applicant: OLYMPUS CORPORATION, Tokyo (JP)

(72) Inventor: Yoko Horino, Tokyo (JP)

(73) Assignee: OLYMPUS CORPORATION, Tokyo (JP)

(21) Appl. No.: 14/283,933

(22) Filed: May 21, 2014

Related U.S. Application Data
(63) Continuation of application No. PCT/JP2012/083436, filed on Dec. 25, 2012.

(30) Foreign Application Priority Data
Dec. 27, 2011 (JP) 2011-285214

(51) Int. Cl. A61K 49/00; A61B 1/04; G01N 33/574

(52) U.S. Cl. CPC A61K 49/0017; G01N 33/57419; A61B 1/043
USPC 424/9.6; 424/9.1; 435/7.23; 530/395; 435/193; 536/23.1; 530/350

(57) ABSTRACT

The present invention provides a method for detecting ACF by analyzing a test region of large intestine tissue at the molecular level. Namely, the present invention relates to a method for detecting aberrant crypt foci (ACF) that comprises detecting an ACF detection marker in a test region of large intestine tissue, by using one or more types of molecules for which ACF-specific expression increases as the ACF detection marker, the molecule being selected from the group consisting of Met, Cdh1, Ctnnb1, and GSTp; an ACF detection marker for detecting the ACF in human-derived large intestine tissue, that is Met, Cdh1, Ctnnb1, or GSTp; and, a method for evaluating risk of colorectal cancer and colorectal adenoma in human subjects based on the results of detecting ACF in a test region of large intestine tissue of the subjects using the aforementioned ACF detection method.

The present application claims priority on the basis of Japanese Patent Application No. 2011-285214, filed in Japan on Dec. 27, 2011, the contents of which are incorporated herein by reference. The present application is a U.S. continuation application based on the PCT International Patent Application, PCT/JP2012/083436, filed on Dec. 25, 2012; the contents of which are incorporated herein by reference.

### BACKGROUND OF THE INVENTION

1. Field of the Invention

The present invention relates to a method for detecting aberrant crypt foci (ACF) by using molecules specifically highly expressed for ACF.

2. Description of the Related Art

Colorectal cancer is the leading cause of death in Japan and the second leading cause of death in the U.S. Approximately 150,000 new cases of colorectal cancer are discovered in the U.S. each year, and more than 50,000 of those cases die annually (as estimated by the American Cancer Society). On the other hand, since colorectal cancer frequently takes several tens of years to progress from a benign tumor to a malignant tumor, early risk assessment and discovery are expected to contribute to favorable prognosis and prevention.

Typical examples of colorectal adenoma and tumor screening methods currently employed include occult blood testing, barium enema, total colonoscopy and sigmoid colonoscopy. However, in the case of occult blood testing, for example, since there are cases in which blood is detected that is caused by factors other than an adenoma or tumor, it cannot be said to have high specificity for colorectal adenomas and tumors, and is susceptible to the occurrence of false positives when used for the purpose of early detection. On the other hand, although barium enemas are able to detect largely formed advanced cancer, it has the disadvantage of having difficulty in detecting small lesions.

In addition, since endoscopic examinations allow visualization of an affected site directly, the examination results are highly reliable and these examinations have been shown to contribute to reductions in the mortality rate and incidence of colorectal cancer. However, since the affected area may be extremely small in the early stages of cancer, there is the problem of such affected areas being difficult to detect by endoscopic examination.

In this manner, since detection techniques for effectively detecting groups at high risk to colorectal cancer and early colorectal cancer have yet to be fully developed, there are many cases in which diagnosis is made only after the disease stage has advanced to a certain degree. Therefore, there is a desire for a highly sensitive and highly specific testing method that enables risk assessment and discovery of colorectal cancer at an early stage either lowly invasively or non-invasively.

A method that is currently attracting attention as a method for detecting colorectal cancer from the stage of an early lesion consists of analyzing at the molecular level using nucleic acid and protein analysis techniques. For example, a genetic predisposition to familial adenomatous polyposis (FAP) is a typical example of a risk factor of colorectal cancer, and the risk of colorectal cancer can be accessed by conducting a genetic analysis on the subject. More recently, among groups having and not having a characteristic genetic predisposition in the manner of FAP, age (50 years or older) and lifestyle factors such as obesity, alcohol consumption and smoking are known to increase the future risk of colorectal cancer. Consequently, molecular abnormalities (epigenetics, expression abnormalities) attributable to lifestyle are attracting attention as a way of predicting future colorectal cancer. In actuality, numerous molecules have been found during the course of genome-wide association studies (GWAS) that are suggested to be involved in colorectal cancer.

Technologies for analyzing nucleic acids present in stool and blood are being developed for the purpose of determining the presence of molecular abnormalities in the large intestine. However, since nucleic acids originating in extremely small lesions are only present in trace amounts, early stage detection of molecular abnormalities is difficult. The reflection of changes in microlesions, having 50 crypts or less, or measuring 1 mm or less in the blood is also difficult in terms of the sensitivity of analytical devices. In addition, stool contains a large amount of intestinal bacteria as well as epithelial cells that have exfoliated from regions other than lesions, and there is a large amount of noise. Consequently, in order to detect early stage colorectal adenomas or colorectal tumors when using stool for the specimen, a superior molecular marker is required that increases in expression level to a greater degree than in normal tissue at an early stage of canceration and tumor development. Thus, the development of a technology for early risk assessment of colorectal cancer by detecting molecular abnormalities at an early stage in the large intestine has yet to be realized.

On the other hand, aberrant crypt foci (ACF) were first reported by Bird, et al. in 1987 as microlesions that are densely stained with methylene blue in the large intestine of rats administered a carcinogenic substance (azoxymethane). ACF constitute the first morphological abnormality that can be detected morphologically (see, for example, Kelloff, et al., 2006, Clinical Cancer Research, Vol. 12, No. 12, pp. 3661-3697), and since accelerated cell proliferation activity and κ-ras mutations are also observed, ACF have been suggested to be involved in the onset of colorectal cancer and colorectal adenoma. Lesions densely stained with methylene blue have also been observed in excised specimens of human large intestine in cancer patients and patients with polyps, and the amount of these lesions has been reported to increase in the order of healthy persons, patients with polyps and cancer patients (see, for example, Takayama, et al., The New England Journal of Medicine, 1998, Vol. 339, No. 18, pp. 1277-1284). On the basis of these findings, ACF is being more frequently used as an indicator in colorectal cancer preventive research.

ACF measuring 1 mm or less in size are difficult to be detected in ordinary endoscopic examinations, and are generally detected using a magnifying endoscope. However, since the use of a magnifying endoscope requires considerable time for the procedure, the opportunities for its use are limited and it is difficult to be used for primary screening of early colorectal cancer. In this manner, development of a technology for evaluating the risk of colorectal cancer by detecting micro ACF microscopically or endoscopically has yet to be realized.

In addition, searches have been made for useful molecular markers in order to analyze ACF at the molecular level. More specifically, reported examples of molecules for which expression levels fluctuate in ACF include cyclin D1, cyclooxygenase 2 (COX2), catenin beta 1 (β catenin), nitric oxide synthase 2, inducible (iNOS); epidermal growth factor receptor (EGFR) and CD44 molecule (CD44). However, each of these markers has been reported on the basis of small-scale studies, and differing from colorectal cancer, there have been no reports relating to evaluation of large-scale studies regarding molecular alterations in ACF lesions.

For example, in an example of prior research on COX2 (see Fichera, et al., Journal of Surgical Research, 2007, Vol. 142, pp. 239-245), since molecular alterations were analyzed using samples collected throughout the large intestine, specificity for ACF was low and molecular alterations that can be ascribed only to ACF were not analyzed in comparison to the surrounding (normal) tissue. In addition, in prior research on cyclin D1 (see Paulsen, et al., Cancer Research, 2005, Vol. 65, pp. 121-129), prior research on iNOS (see Xu, et al., World Journal of Gastroenterology, 2003, Vol. 9, No. 6, pp. 1246-1250) and prior research on CD44 (see Boon, et al., Cancer Research, 2002, Vol. 62, pp. 5126-5128), since only immunostaining data was analyzed, this research lacks quantitativeness and specificity and has not been able to be applied to actual medical treatment. Moreover, in prior research on iNOS (see Takahashi, et al., Carcinogenesis, 2000, Vol. 21, No. 7, pp. 1319-1327) and prior research on EGFR (see Cohen, et al., Cancer Research, 2006, Vol. 66, pp. 5126-5128), analyses were targeted at comparatively large ACF of 50 crypts or so, while analyses were not conducted on molecular alterations of early stage micro ACF. In this manner, although several molecules are known which have been suggested as having the possibility of being able to be used as ACF marker molecules, none of these are considered to be reliable enough to warrant clinical application.

In other words, the analysis of molecules demonstrating alterations in expression level at the stage of microlesions typically represented by ACF having 50 crypts or less, or micro ACF measuring 1 mm or less (namely, at an early stage), and the simple assessment of the future risk of colorectal cancer by molecular biological determination, have yet to be realized.

### SUMMARY OF THE INVENTION

As a result of conducting extensive research to solve the aforementioned problems, the inventors of the present invention found that, in human-derived colorectal tissue, expression levels of Met (met proto-oncogene), Cdh1 (Cadherin 1), Ctnnb1 (Catenin beta), and GSTp (glutathione S-transferase pi) increased more in micro ACF measuring 1 mm or less or having 50 crypts or less, than in normal tissue, thereby leading to completion of the present invention.

Namely, the present invention provides:

(1) a method for detecting aberrant crypt foci (ACF), comprising: detecting an ACF detection marker in a test region of large intestine tissue, by using one or more types of molecules for which ACF-specific expression increases as the ACF detection marker, the molecules being selected from the group consisting of Met, Cdh1, Ctnnb1, and GSTp;

(2) the method for detecting ACF described in (1) above, wherein the test region comprises a region where ACF are suspected;

(3) the method for detecting ACF described in (2) above, comprising comparing the amount of the ACF detection marker in the test region with the amount of the ACF marker in a region of normal tissue in the same large intestine tissue as the test region;

(4) the method for detecting ACF described in any of (1) to (3) above, wherein the test region is a specimen collected from the body;

(5) the method for detecting ACF described in any of (1) to (3) above, wherein detection of the ACF marker is carried out in vivo;

(6) the method for detecting ACF described in any of (1) to (5) above, wherein the detecting is done by fluorescent labeling;

(7) the method for detecting ACF described in any of (1) to (6) above, wherein the ACF marker is mRNA or proteins;

(8) the method for detecting ACF according to any of (1) to (7), wherein the detection is done by fluorescently labeling the ACF detection marker in the test region with a fluorescently labeled probe or specific antibody, and then conducting optical detection using an endoscope or gastrointestinal video scope;

(9) an ACF detection marker, which is Met, Cdh1, Ctnnb1 or GSTp; and

(10) a method for evaluating risk of colorectal cancer and colorectal adenoma in human subjects based on the results of detecting ACF in a test region of large intestine tissue of the subjects using the ACF detection method described in any of (1) to (8) above.

### BRIEF DESCRIPTION OF THE DRAWINGS

FIG. 1 depicts graphs showing a distribution of the relative expression levels of Met in control samples (samples of normal tissue) and ACF samples (samples of ACF sites) in Example 1.

FIG. 2 depicts graphs showing a distribution of the expression levels of Cdh 1 in each sample, after being normalized with the expression level of 18SrRNA, in Example 1.

FIG. 3 depicts graphs showing a distribution of the expression levels of Ctnnb1 in each sample, after being normalized with the expression level of 18SrRNA, in Example 1.

FIG. 4 depicts graphs showing a distribution of the expression levels of GSTp1 in each sample, after being normalized with the expressin level of 18SrRNA, in Example 1.

FIG. 5 depicts graphs showing a distribution of the expression levels of EGFR in each sample, after being normalized with the expression level of 18SrRNA, in Example 1.

FIG. 6 depicts graphs showing a distribution of the expression levels of NOS2 in each sample, after being normalized with the expression level of 18SrRNA, in Example 1.

FIG. 7 depicts graphs showing a distribution of the expression levels of CD44 in each sample, after being normalized with the expression level of 18SrRNA, in Example 1.

FIG. 8 depicts graphs showing a distribution of the expression levels of Ctsb in each sample, after being normalized with the expression level of 18SrRNA, in Example 1.

FIG. 9 depicts graphs showing a distribution of the expression levels of PCNA in each sample, after being normalized with the expression level of 18SrRNA, in Example 1.

FIG. 10 depicts graphs showing a distribution of the expression levels of Fzd 1 in each sample, after being normalized with the expression level of 18SrRNA, in Example 1.

FIG. 11 depicts graphs showing a distribution of the expression levels of COX2 in each sample, after being normalized with the expression level of 18SrRNA, in Example 1.

FIG. 12 depicts images of HE staining and immune-histochemical staining of sample ACF lesions in Example 1.

FIG. 13A is images of ACF lesions, which is a surgically excised sample of a human colon, fluorescently stained with a GSTp1 fluorescent prove and subjected to imaging with a microscope, in Example 2.

FIG. 13B is images of ACF lesions, which is a surgically excised sample of a human colon, fluorescently stained with a GSTp1 fluorescent prove and subjected to imaging with an endoscope, in Example 2.

FIG. 14A is images of a colon cancer site, which is a surgically excised sample of a human colon, fluorescently stained with a GSTp1 fluorescent prove and subjected to imaging with a microscope, in Example 2.

FIG. 14B is images of a colon cancer site, which is a surgically excised sample of a human colon, fluorescently stained with a GSTp1 fluorescent prove and subjected to imaging with an endoscope, in Example 2

FIG. 15 is a graph showing the results by comparing fluorescence intensities in ACF lesions and normal region, obtained from a fluorescence image of a surfically exised sample of a human colon, fluorescently stained with a GSTp1 fluorescent probe, in Example 2.

### DETAILED DESCRIPTION OF THE PREFERRED EMBODIMENTS

In the present invention and the description of the present application, molecules for which ACF-specific expression increases refer to molecules for which gene expression levels increase in ACF more than in surrounding normal tissue in the large intestine tissue of the same individual.

In addition, in the present invention and the description of the present application, large intestine indicates a region that includes the appendix, colon, rectum and anus, while large intestine tissue indicates tissue that includes large intestine mucous membrane and large intestine epithelium.

In the present invention and description of the present application, the diameter of a region indicates diameter in the case the region is a circle or oval (long axis in the case of an oval), the diameter of an approximate circle when the region is approximated to a circular shape in the case the region is of a shape other than a circle or oval, or the diameter of an approximate oval in the case of having approximated the shape of the region to that of an approximate oval.

The ACF detection method of the present invention is characterized by detecting an ACF marker present in a test region of large intestine tissue, using one of more types of molecules for which ACF-specific expression increases selected from the group consisting of Met, Cdh1, Ctnnb1, and GSTp. Although GSTp may have a plurality of isoforms, these isoforms are only required to be those that are expressed in large intestine tissue, and only one type of isoform may be detected or a plurality of isoforms may be detected. All of these four types of molecules for which ACF-specific expression increases are molecules for which expression levels increase in micro ACF, specifically those measuring 1 mm or less or having 50 crypts or less, more than in surrounding normal tissue. Consequently, these four types of molecules for which ACF-specific expression increases are clinically useful marker molecules for detecting ACF and particularly for detecting micro ACF, and by using the expression levels of these molecules for which ACF-specific expression increases as indicators (using the molecules as the ACF detection markers), comparatively large ACF (namely, ACF in which morphological abnormalities have progressed) as well as early micro ACF can be accurately detected.

In the ACF detection method of the present invention, at least one type of the aforementioned four types of molecules for which ACF-specific expression increases may be detected, or two or more types may be detected for a single test region.

Although there are no particular limitations on the test region where expression levels of molecules (the ACF detection markers) for which ACF-specific expression increases are detected provided it is a region of human large intestine tissue, it may be a region where ACF are suspected (suspect ACF region). An example of a suspect ACF region is a region of large intestine tissue densely stained by methylene blue stain. Although regions other than ACF also end up being stained by methylene blue staining, in the ACF detection method of the present invention, ACF can be accurately detected by using the expression levels of molecules for which ACF-specific expression increases as indicators. Other examples of suspect ACF regions include regions in which morphological abnormalities are observed by endoscopic observation, microscopic observation or image analysis and the like.

Dimension of the test region in the ACF detection method of the present invention is not specifically limited, for example, it can be properly determined by the dimension etc. of a suspected ACF region. However, the higher the ratio of the suspected ACF region to the test region, the better. If the ratio of a normal region to a test region is too high, then it becomes difficult to detect the difference between the amount of molecules for which ACF-specific expression increases in the suspected region and the amount of molecules for which ACF-specific expression increases in the normal tissue, even if the suspected ACF region is actually the ACF. For example, if any suspected regions having a diameter of 1 mm or less are to be included; a diameter of suspected region may be 1 mm or less, the diameter may be less than 1 mm, and the diameter may be 0.5 mm or less. If any suspected regions having 50 crypts or less are to be included; dimension of the suspected region may be 50 crypts or less, it may be less than 50 crypts, and it may be 25 crypts or less.

The four types of molecules for which ACF-specific expression increases targeted for detection in the present invention are all molecules for which gene expression levels increase in ACF more than in normal tissue. Consequently, in the present invention, a relatively large ACF, for example ACF having 100 to 150 crypts, can be suitably detected as in the case of detecting micro ACF having a diameter of 1 mm or less.

The molecules for which ACF-specific expression increases that are detected in the ACF detection method of the present invention are only required to be molecules that reflect gene expression level, and may be mRNA or proteins. Namely, the ACF detection method of the present invention enables detection of ACF in a test region by acquiring information at the RNA level or protein level on molecules for which ACF-specific expression increases in the test region.

The method used to detect each molecule for which ACF-specific expression increases is only required to be a method in which detection results are dependent on the amount or concentration of each molecule in the test region, and can be suitably selected and used from among known methods used to detect mRNA or protein in a specimen. In particular, the methods used to detect molecules for which ACF-specific expression increases may be carried out with a method used for expression analysis. Each method can be carried out in accordance with ordinary methods.

In the case ACF are contained in a test region, the amount of molecules for which ACF-specific expression increases in the test region increases more than in normal tissue in large intestine tissue. Consequently, ACF can be detected in a test region by comparing the amount of molecules for which ACF-specific expression increases present in the test region with the number of molecules for which ACF-specific expression increases present in normal tissue in large intestine tissue. Namely, in the case the amount of molecules for which ACF-specific expression increases in a test region is greater than that in normal tissue, ACF can be judged to be contained in the test region, while in the case that amount is equal to or lower than the amount in normal tissue, ACF can be judged to not be contained in the test region. Comparison of the amounts of molecules for which ACF-specific expression increases between a test region and a normal tissue region may be carried out per unit surface area or unit volume of each region, or may be carried out per unit amount of nucleic acid or unit amount of protein contained in each region.

In the present invention, a test region and normal tissue surrounding that test region may be compared in large intestine tissue of the same individual. Although there are individual differences in the expression level of each molecule for which ACF-specific expression increases, effects attributable to individual differences can be eliminated by comparing within the same individual.

Molecules for which ACF-specific expression increases may not be detected in normal tissue and may only be detected in ACF depending on the type and sensitivity of the detection method used. In this case, ACF are judged to be contained in a test region in the case molecules for which ACF-specific expression increases were detected in the test region, while ACF are judged to not be contained in the test region in the case molecules for which ACF-specific expression increases were not detected.

In the case an ACF detection marker is mRNA, that is, when the an expression level of the molecule for which ACF-specific expression increases is to be determined at an RNA level; examples of methods used to detect each molecule for which ACF-specific expression increases include a method consisting of utilizing a nucleic acid amplification reaction using primers specific to each molecule, and a method consisting of utilizing a hybridization reaction using probes specific for each molecule. An example of a method that utilizes a nucleic acid amplification reaction consists of detecting a molecule for which ACF-specific expression increases in a test region, or quantitatively measuring the amount thereof in a normal tissue region to a degree that allows comparison, by synthesizing cDNA from RNA contained in the test region by a reverse transcription reaction followed by carrying out a nucleic acid amplification reaction such as RT-PCR using the resulting cDNA as a template. Extraction of RNA from the test region, the reverse transcription reaction, and the RT-PCR or other nucleic acid amplification reaction can be suitably selected and carried out from among techniques known in the applicable technical fields.

When using a method that utilizes a nucleic acid amplification reaction, an amplified molecule for which ACF-specific expression increases can be fluorescently labeled and quantitatively detected by using a fluorescent intercalator or primer labeled with a fluorescent substance. On the other hand, when using a method that utilizes hybridization, a molecule for which ACF-specific expression increases can be fluorescently labeled and quantitatively detected in a test region by using a probe labeled with a fluorescent substance or a probe that has been modified so as to emit fluorescent light only after having hybridized.

In the case an ACF detection marker is a protein, that is, when an expression level of the molecule for which ACF-specific expression increases is to be determined at a protein level; each molecule for which ACF-specific expression increases can be detected by an immunochemical technique using an antibody that specifically recognizes each molecule (specific antibody). More specifically, after bonding a specific antibody of each molecule labeled with a marker to the molecule present in a test region, a molecule for which ACF-specific expression increases can be fluorescently labeled and quantitatively detected in the test region by measuring a signal from the marker. Labeling with a marker may be carried out by bonding the marker directly to a specific antibody of each molecule, or by bonding to a secondary antibody that specifically bonds with that specific antibody. The marker can be suitably selected and used from among markers commonly used in antigen-antibody reactions or those used when detecting for the presence or absence of bonding of two molecules. Examples of such markers include fluorescent substances, magnetic substances and radioisotopes. Fluorescent substances may be used as markers from the viewpoints of high sensitivity and safety. The antigen-antibody reaction can be carried out in accordance with ordinary methods. In addition to immunochemical techniques, a probe that specifically indicates activity of a molecule for which ACF-specific expression increases can be used, and a molecule for which ACF-specific expression increases in the form of a protein can be detected by detecting the probe.

Detection of a molecule for which ACF-specific expression increases can be carried out on a specimen collected from the body. For example, a sample of a test region containing a suspect ACF region can be collected microscopically from a sample obtained by excising large intestine tissue collected by surgically excising a partial region of large intestine tissue of the body. At this time, a sample of a normal tissue region, and for example, a normal tissue surrounding the test region, is collected from the same surgically excised large intestine tissue sample as necessary. In addition, a sample of a test region (biopsy sample) can be collected directly from the body by preliminarily staining large intestine tissue of the body with methylene blue and then surgically excising the densely stained region. A sample of normal tissue surrounding the test region can also be collected from the body in the same manner as the surgically excised sample of large intestine tissue. Samples of a test region and normal tissue region collected in this manner are then used to detect molecules for which ACF-specific expression increases. Methylene blue staining of large intestine tissue in vivo can be carried out in accordance with ordinary methods.

Detection of a molecule for which ACF-specific expression increases can also be carried out in vivo. For example, a labeled probe specific for a molecule for which ACF-specific expression increases, a specific antibody of a molecule for which ACF-specific expression increases labeled directly or indirectly, or a specific labeled probe that indicates activity of a molecule for which ACF-specific expression increases, is coated or sprayed onto a region containing a test region in the large intestine of the body, and after allowing the probe or specific antibody to bond to the molecule for which ACF-specific expression increases present in the region, thereby labeling the molecule for which ACF-specific expression increases, the molecule for which ACF-specific expression increases can be detected by detecting the label.

In the case of detecting a molecule for which ACF-specific expression increases in vivo, the molecule for which ACF-specific expression increases may be detected by fluorescent labeling. More specifically, a molecule for which ACF-specific expression increases is first fluorescently labeled using a probe or specific antibody labeled with a fluorescent substance. Subsequently, fluorescence emitted from the label is optically detected using a devise which enables optical detection of inside of colon, such as an endoscope or gastrointestinal video scope, to obtain a fluorescent image. A molecule for which ACF-specific expression increases can then be detected both with high sensitivity and quantitatively by analyzing the resulting fluorescent image.

Detection of a molecule for which ACF-specific expression increases in vivo can be carried out easily and efficiently by using a fluorescent endoscope. More specifically, an endoscopy system can be used, for example, that is partially inserted into a body cavity of the body to acquire images of an imaging target within the body cavity, and is provided with agent discharge means for discharging toward the imaging target a sensitive fluorescent agent that bonds or reacts with a specific substance present in the imaging target or a fluorescent agent that is accumulated in the imaging target, discharge control means for controlling the agent discharge means, a light source unit that emits excitation light for exciting the fluorescent agent and irradiation light having spectral properties that differ from those of the excitation light, optics for allowing the excitation light and irradiation light from the light source unit to propagate towards the imaging target, and imaging means provided at a site that is inserted into the body cavity and is capable of capturing images of fluorescent light radiated from the imaging target due to the excitation light and light of a frequency band differing from the fluorescent light radiated from the imaging target due to the irradiation light (see, Japanese Unexamined Patent Application, First Publication No. 2007-229054). A probe or specific antibody specific for a molecule for which ACF-specific expression increases that has been labeled with a fluorescent substance is used for the fluorescent agent.

In the ACF detection method of the present invention, the sample provided for detection of a molecule for which ACF-specific expression increases is only required to be that which originates in the large intestine of animal human, and may be a sample of large intestine tissue directly excised from a human subject, large intestine tissue collected from a living body or in vitro-cultured cells constituting the tissue. ACF can be also detected in any animal species in which the expression levels of Met, Cdh1, Ctnnb1 or GSTp are increased in ACF than in normal tissue as in the case of a human, by detecting at least one type of molecules for which ACF-specific expression increases selected from the group consisting of Met, Cdh1, Ctnnb1, and GSTp in a suspected region of the animal-derived large intestine tissue.

Information as to whether or not the amount of a molecule for which ACF-specific expression increases in a test region is greater than the amount in normal tissue is useful when judging whether or not ACF are present in the test region. Accordingly, detection results obtained according to the ACF detection method of the present invention are useful as information provided for diagnosing ACF.

Since ACF have a high probability of progressing to colorectal cancer in the future, detection results obtained according to the ACF detection method of the present invention serve as extremely useful information when assessing the risk of existing colorectal cancer, and during lowly invasive assessment of the risk of future colorectal cancer at an early stage. For example, according to the ACF detection method of the present invention, in the case in which the amount of a molecule for which ACF-specific expression increases in a test region is greater than the amount in a surrounding normal tissue region in large intestine tissue of a subject, and ACF have been detected in the test region, the subject can be assessed has having a high risk of the onset of colorectal cancer and colorectal adenoma in the future. Conversely, in the case the amount of a molecule for which ACF-specific expression increases in a test region is equal to or less than the amount in a surrounding normal tissue region, and ACF is not detected in the test region, the subject can be assessed has having a low risk for the onset of colorectal cancer and colorectal adenoma in the future.

### EXAMPLES

Although the following provides a more detailed explanation of the present invention by indicating examples thereof, the present invention is not limited to the following examples.

### Example 1

The gene expression levels of all 11 types of candidate molecules consisting of Met, Cdh1, Ctnnb1, GSTp1, EGFR, iNOS (NOS2), CD44, Fzd1, Ctsb (cathepsin B), PCNA (proliferating cell nuclear antigen), and COX2 were compared between a surrounding normal tissue region and a suspect ACF region in the same individual to identify those candidate molecules for which gene expression levels increased specifically for ACF.

More specifically, a sample collected only from a region confirmed microscopically to an ACF site and a sample collected from normal tissue colonoscopically obtained from a subject were respectively prepared from biopsy large intestine samples collected from the subject who had a colonoscopic examination; and the expression levels of each molecule in each sample were measured and compared. The following provides a description of the details thereof.

The large intestine mucosal tissue obtained from the rectum was microscopically observed, and excised only a site confirmed to be the ACF site as a sample. At this time, micro ACF measuring 1 mm or less were selected and collected, using a commercially available biopsy instrument of the minimum size (diameter: 1 mm). On the other hand, only the site confirmed to be normal by magnifying endoscopy was excised as a control sample. A plurality of samples for both ACF sample and control sample was obtained from a single subject.

To prevent decomposition of nucleic acids, the ACF sample and the control sample were immersed in RNAlater (QIAGEN Inc.) soon after biopsy. Then, using MagnaLyser (Roche Inc.) and QIAGEN RNase mini kit (QIAGEN Inc.), total RNA was extracted, followed by DNase (Invitrogen Inc.) treatment to digest remaining DNA. In a reaction solution to which RNA, the quality of which was confirmed to have RIN 6 or more, was added; an RT reaction was carried out for 60 minutes at 37° C. For the pre-amplification reaction, a pre-amplification reaction was carried out for a low number of cycles using a primer set for amplifying each candidate molecule by using the resulting cDNA as template. The commercially available primers shown in Table 1 (Applied Biosystems Inc.) were respectively used for the primer sets. More specifically, 7 μL of reaction solution following the RT reaction, 12.5 μL of solution obtained by preliminarily mixing each primer set, 25 μL of nucleic acid amplification reagent (Taqman Gene Expression Master Mix, Applied Biosystems Inc.) and 5.5 μL of ultrapure water were each added to prepare a reaction solution having a final volume of 50 μL. Each reaction solution was placed in a PCR device (Eppendorf Corp.), and after heat treating for 10 minutes at 95° C., PCR was carried out for 14 cycles of a thermal reaction consisting of 15 seconds at 95° C. and 4 minutes at 60° C. Following reaction, the reaction liquid was diluted 20 fold and provided for use as real-time PCR sample.

TABLE 1

| Candidate Molecule | Product Name |
|---|---|
| Met | Hs01565584_m1 |
| Cdh1 | Hs01023894_m1 |
| Ctnnb1 | Hs00355049_m1 |
| GSTp1 | Hs00943351_g1 |
| EGFR | Hs01076078_m1 |
| NOS2 | Hs01085529_m1 |
| CD44 | Hs01075861_m1 |
| Ctsb | Hs00947433_m1 |
| PCNA | Hs00696862_m1 |
| Fzd1 | Hs00268943_s1 |
| COX2 | Hs00153133_m1 |

Real-time PCR was then carried out using the pre-amplified cDNA as template followed by detection of the expression products (mRNA) of each candidate molecule. More specifically, after dispensing 5 μL aliquots of each pre-amplified cDNA into a 0.2 mL 96-well plate, 4 μL of ultrapure water, 10 μL of nucleic acid amplification reagent (Taqman Gene Expression Master Mix, Applied Biosystems Inc.) and 1 μL of primer probe set were added to each well to prepare PCR reaction solutions. The 96-well plate was placed in a real-time PCR device (Applied Biosystems Inc.), and after heat treating for 2 minutes at 50° C. and 10 minutes at 95° C., 40 cycles of a thermal reaction consisting of 15 seconds at 95° C. and 1 minute at 60° C. were carried out, fluorescence intensity was measured over time.

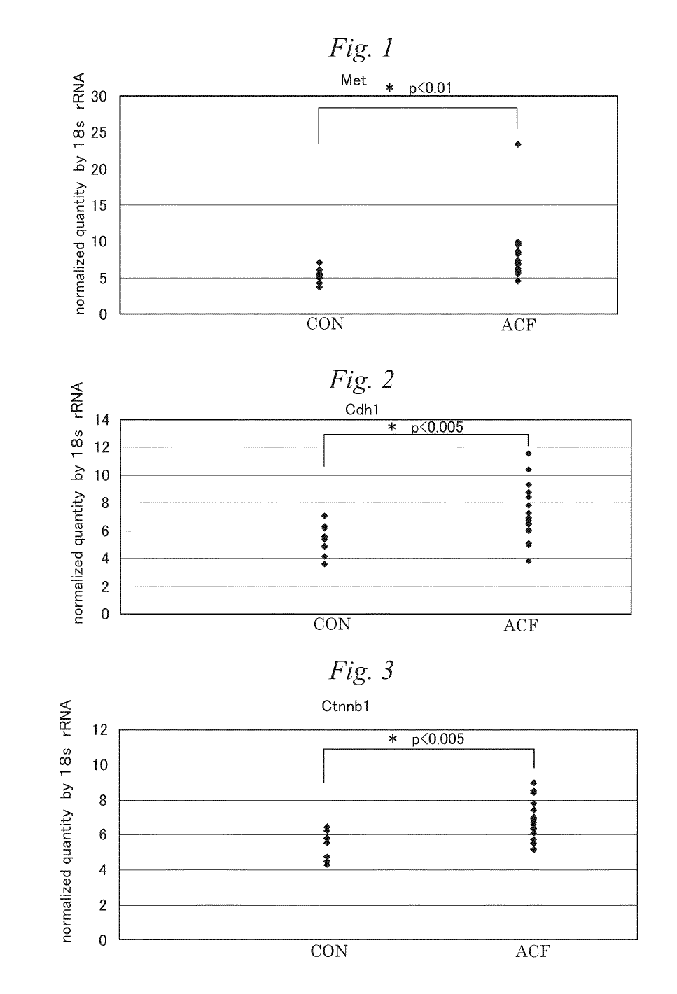

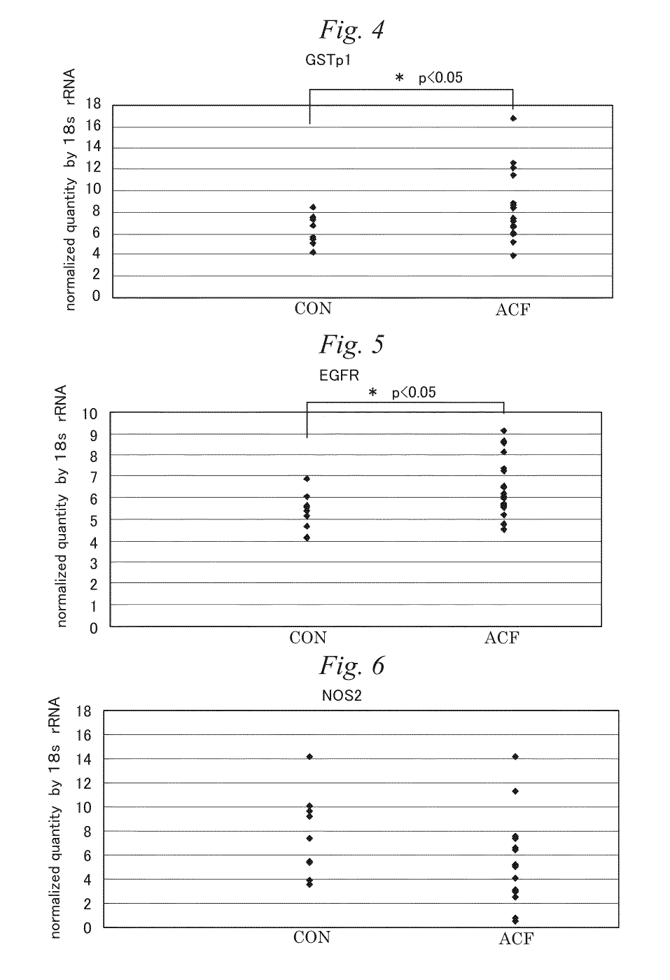

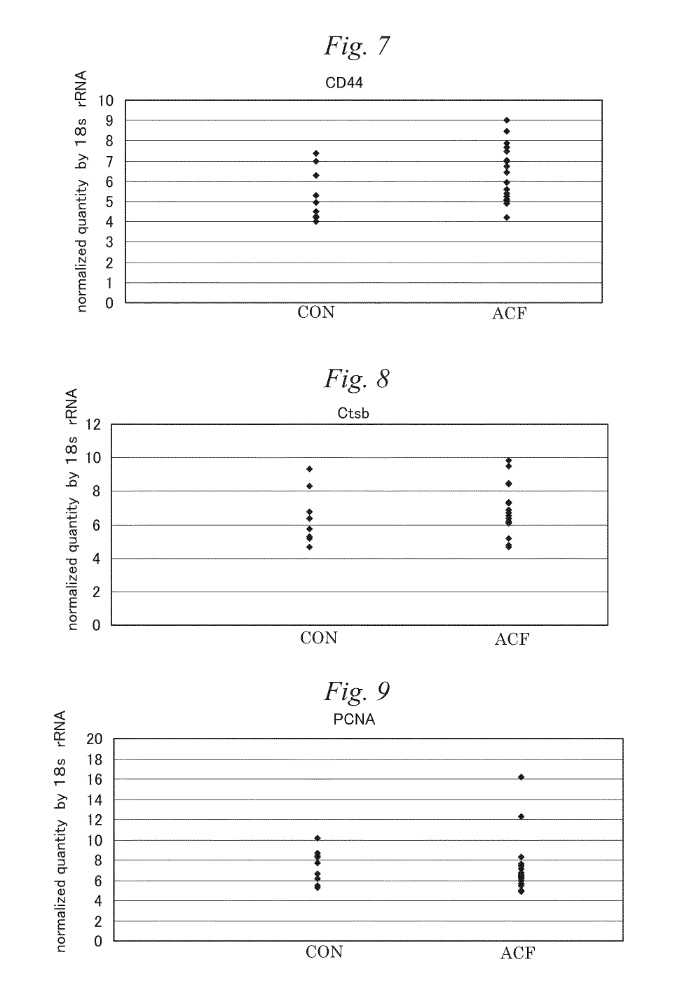

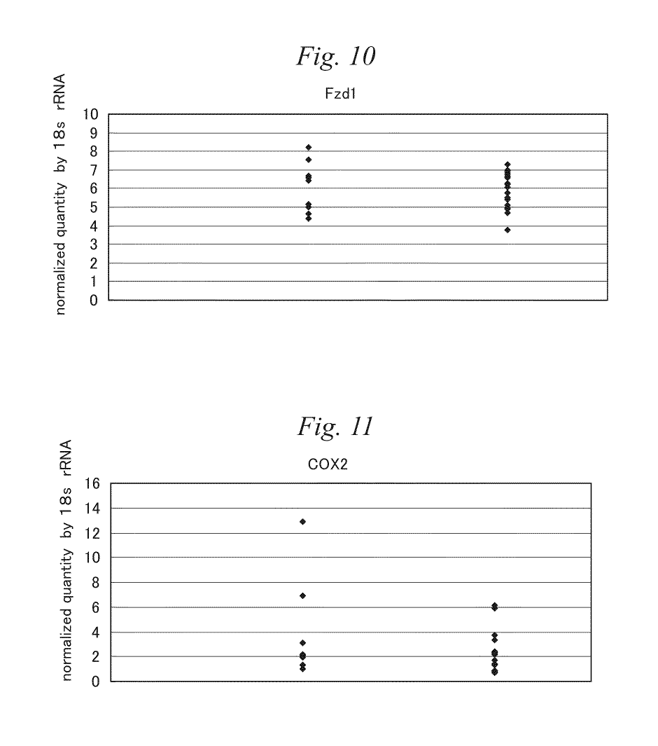

The results of measuring fluorescence intensity were analyzed, and the gene expression levels of the candidate molecules in the RNA recovered from each sample were calculated. Each gene expression level was normalized based on the expression level of 18S rRNA. The results of a distribution of the normalized expression level for each candidate molecule in each sample were summarized in FIGS. 1 to 11, respectively. In each of the graphs, “CON” indicates the result for the control sample and “ACF” indicates the result for the ACF sample.

As a result, when comparisons were made between expression levels in normal tissue and expression levels in ACF in the same individual, expression levels were significantly increased in the ACF lesions (ACF samples) in comparison with surrounding normal tissue (control samples) for the 5 types of molecules of Met, Cdh1, Ctnnb1, GSTp1, and EGFR. In other words, these five types of molecules were determined to demonstrate increased gene expression levels specific for ACF, therefore they can be useful for a marker to detect ACF in human-derived large intestine tissue.

Furthermore, when the expression level in the control sample is set to be 1, the relative expression levels, especially that of Met, in the ACF samples obtained from the same subject was found to be: 1.3 or more in 60% or more of the ACF sample, 1.5 or more in 50% or more of the ACF sample. Therefore, it is expected that this can be sufficiently applicable to an AFC detection marker in the examination such as clinical tests, for which a high reliability is required.

For the remaining 6 types of the molecules, there was not a significant difference in expression level observed between the ACF sample and the control sample. Although only about 20% of the ACF samples showed the relative expression level of 1.3 or more, about 75% of these showed a relative expression level of 1.5 or more. In other words, it suggested that COX2 is not sensitive enough in the ACF detection, but it can be used as a marker with a good specificity.

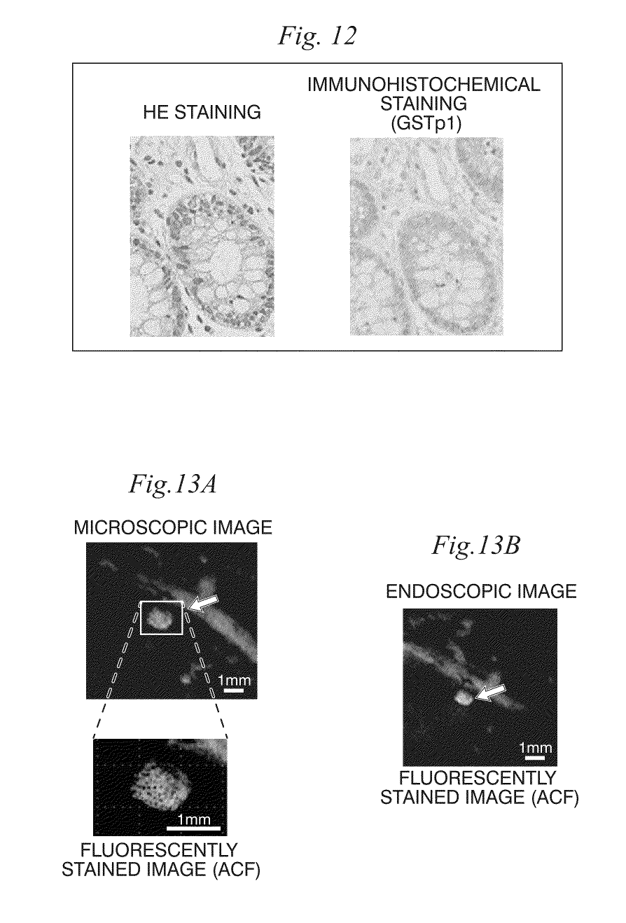

Furthermore, the expression of GSTp1 protein in the ACF lesions of the large intestine biopsy sample obtained from a subject who had a colonoscopic examination was confirmed by immunostaining. Specifically, HE staining, and immunohistochemical staining using GSTp1 protein-specific antibody (a primary antibody: GST-π rabbit polyclonal antibody (product No.: MSA-102, Assay Design Inc.) a secondary antibody: peroxidase-labeled anti-rabbit Ig goat polyclonal antibody (product Name: Envision Detectin Reagent, Product No.: K5027, Dako Inc.)) were conducted. As a result, GSTp1 protein expression was confirmed in the ACF lesions as shown in FIG. 12.

### Example 2

Based on the results of Example 1, it was confirmed that high levels of expression in mRNA level and protein level were seen in human large intestine ACF. This led us to conduct a study to see a probe reaction in human large intestine excised sample, using a GSTp1 fluorescent probe. The GSTp1 fluorescent probe used in this study has a substrate structure of GSTp1, therefore, it is a fluorescent probe for detecting an activity of an enzyme whose fluorescent characteristics changes by the reaction with GSTp1, and having fluorescent characteristics of an excitation wavelength of 490 nm and absorption wavelength of 520 nm (see Fujikawa et al., Journal of the American Chemical Society, 2008, Vol. 130, pp. 14533-14543).

More specifically, the GSTp1 fluorescent probes were sprayed onto surgically excised human large intestine sample, and fluorescence observation was conducted with a microscope and an endoscope. In more detail, Colonoscopic examination was carried out to a subject who had been diagnosed to be colon cancer or ulcerative colitis before receiving a removal operation, thereby confirming the lacation of ACF lesion by staining with methylene blue. Next, the excised sample was washed with warm PBS immediately after the removal operation, longitudinally dissected, and then a GSTp11 fluorescent probe solution was sprayed thereon, let it react at 37° C. for 20 min in a dark room. After the probe reaction, the tissue was washed with warm PBS, and fluorescent observation and evaluation were microscopically and endoscopically conducted on the ACF lesions and colon cancer. The microscopy was conducted with a stereo microscope MVX10 (Olympus, Ltd.), Band-Pass Filter of 460-490 nm as EX (excitation) filter, IF Filter of 510 nm or this in combination with 510-550 nm Band-Pass Filter as EM (emission) filter. In the endoscopy, an endoscope with a light source of a rigid endoscope whose excitation wavelength being set to 465-490 nm, and Band-Pass Filter of 510-550 nm as fluorescent filter was used.

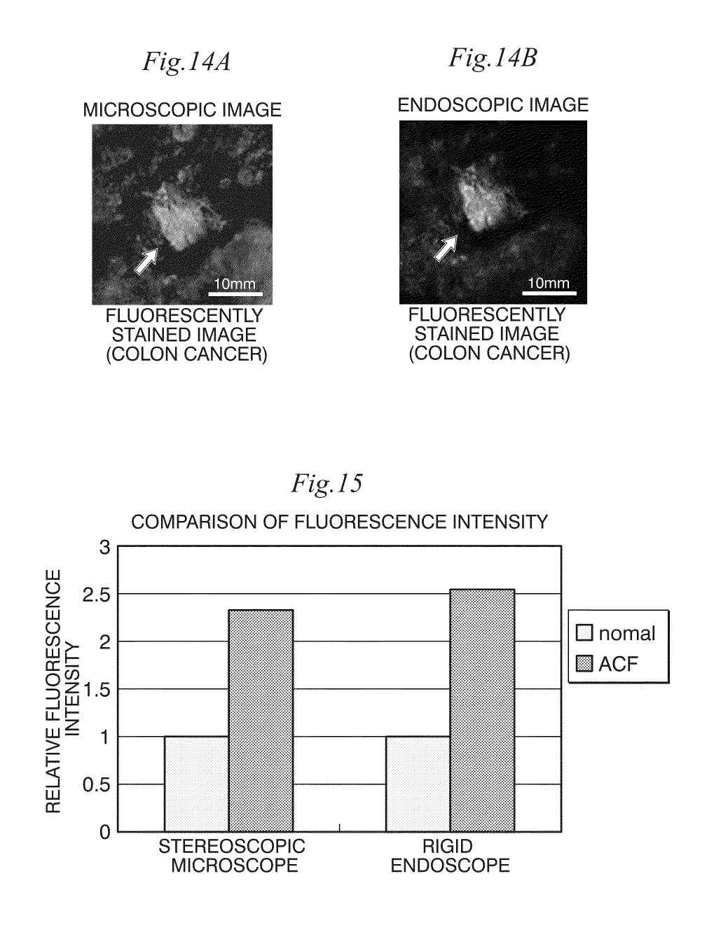

FIG. 13A and FIG. 13B show a fluorescence imaging of the ACF lesions by microscopy, and a fluorescence imaging of the ACF lesions by endoscopy, respectively. In FIG. 13A and FIG. 13B, a portion indicated with a white arrow is the site which is stained with methylene blue, which is the ACF lesion. FIG. 14A and FIG. 14B show a fluorescence imaging of colon cancer sites by microscopy and a fluorescence imaging of the colon cancer sites by endoscopy, respectively. In FIG. 14A and FIG. 14B, a portion indicated with a white arrow is the colon cancer site. As shown in FIG. 13 and FIG. 14, compared to the surrounding normal regions, fluorescence intensities in the ACF lesions and the colon cancer sites are increased, and strong probe reactions were recognized.

Furthermore, results obtained with image analysis are shown in FIG. 15. As a result, the fluorescence intensity at the ACF sites within the same individual have relative values of 2.3 in microscopy and 2.5 in endoscopy, compared to the fluorescence intensity in normal sites. Based on the results thus obtained, GSTp1 fluorescent probe is preferentially incorporated into the ACF lesions of a human large intestine tissue.

Based on these results, it is clear that use of GSTp1 fluorescence probe enables the detection of ACF lesions by microscopic imaging and endoscopic imaging. Moreover, it is suggested that use of GSTp1 fluorescence probe enables in vivo detection of ACF lesions, since sufficient fluorescence intensity and contrast to discern ACF lesions were obtained not only in microscopy but also in endoscopy.

In the ACF detection method according to the examples of the present invention, human ACF can be detected using a molecular biological technique. In particular, the ACF detection method according to the examples of the present invention is able to accurately detect micro ACF measuring 1 mm or less, or having 50 crypts or less.

Since ACF are considered to be indicators of colorectal cancer and colorectal adenoma, the ACF detection method according to the examples of the present invention is useful for early detection of colorectal cancer and colorectal adenoma, and the assessment of the risk thereof.

1. A method for detecting aberrant crypt foci (ACF), comprising:
detecting an ACF detection marker in a test region of large intestine tissue, by using one or more types of molecules for which ACF-specific expression increases as the ACF detection marker, the molecules being selected from the group consisting of Met, Cdh1, Ctnnb1, and GSTp.

2. The method for detecting ACF according to claim 1, wherein the test region comprises a region where ACF are suspected.

3. The method for detecting ACF according to claim 2, comprising:
comparing the amount of the ACF detection marker in the test region with the amount of the ACF detection marker in a region of normal tissue in the same large intestine tissue as the test region.

4. The method for detecting ACF according to claim 1, wherein the test region is on a specimen collected from the body.

5. The method for detecting ACF according to claim 1, wherein detection of the ACF detection marker is carried out in vivo.

6. The method for detecting ACF according to claim 1, wherein the detection is done by fluorescent labeling.

7. The method for detecting ACF according to claim 1, wherein the ACF detection marker is mRNA or proteins.

8. The method for detecting ACF according to claim 1, wherein the detection is done by fluorescently labeling the ACF detection marker in the test region with a fluorescently labeled probe or specific antibody, and then conducting optical detection using an endoscope or gastrointestinal video scope.

9. An ACF detection marker, which is Met, Cdh1, Ctnnb1 or GSTp.

10. A method for evaluating risk of colorectal cancer and colorectal adenoma in human subjects based on the results of detecting ACF in a test region of large intestine tissue of the subjects using the ACF detection method according to claim 1.
